(19) United States
(12) Patent Application Publication
(10) Pub. No.: US 2023/0220121 A1
(43) Pub. Date: Jul. 13, 2023

(54) USE OF ALKANOLAMINES FOR LIGNIN EXTRACTION IN THE PRETREATMENT OF BIOMASS

(71) Applicants: National Technology and Engineering Solutions of Sandia, LLC, Albuquerque, NM (US); The Regents of the University of California, Oakland, CA (US)

(72) Inventors: Ezinne ACHINIVU, Alexandria, VA (US); John M. GLADDEN, Alameda, CA (US); Blake A. SIMMONS, San Francisco, CA (US)

(21) Appl. No.: 17/925,266

(22) PCT Filed: Apr. 29, 2021
(86) PCT No.: PCT/US2021/030023

§ 371 Date: Nov. 14, 2022

Related U.S. Application Data
(60) Provisional application No. 63/023,770, filed on May 12, 2020.

(51) Int. Cl. C08B 37/00; C12P 19/14

(52) U.S. Cl. CPC C08B 37/0003; C12P 19/14

(57) ABSTRACT

The present invention provides for a method to produce a sugar compound from a biomass, the method comprising: (a) providing a first mixture comprising a solubilized biomass and an alkanolamine; (b) recovering at least part of the alkanolamine from the first mixture in order to separate the at least part of the alkanolamine from the first mixture; (c) optionally introducing an enzyme and/or a microbe to the first mixture such that the enzyme and/or microbe produce a sugar from the solubilized biomass; and, (d) optionally the sugar is separated from the first mixture.

### CROSS-REFERENCE TO RELATED APPLICATIONS

This application claims priority to U.S. Provisional Pat. Application Ser. No. 63/023,770, filed on May 12, 2020, which is hereby incorporated by reference.

### STATEMENT OF GOVERNMENTAL SUPPORT

The invention was made with government support under Contract Nos. DE-AC02-05CH11231 awarded by the U.S. Department of Energy. The government has certain rights in the invention.

### FIELD OF THE INVENTION

The present invention is in the field of using alkanolamines for biomass pretreatment.

### BACKGROUND OF THE INVENTION

Biofuels and bioproducts derived from sustainable feedstocks are considered a potential solution to address the challenges associated with human population growth. For efficient biofuel production, the biochemical conversion of lignocellulosic biomass has been frequently discussed in terms of process optimization as well as the reaction mechanism of various thermochemical processing (e.g., pretreatment) and biochemical conversion (e.g.. enzymatic hydrolysis and fermentation). Current challenges to the realization of an affordable and scalable biomass conversion technology are those associated with complicated process designs, difficulties associated with efficient solvent recycle, and water consumption.

U.S. Pat. No. 9,011,640 discloses a method for obtaining raw pulp by removal of lignin from a lignocellulosic biomass in the form of plants and/or plant parts, and wherein the lignocellulosic biomass does not originate from wood, comprising the steps of: digesting the lignocellulosic biomass in a digester at a digestion temperature of less than about 170° C. in a digestion medium to thereby dissolve lignin from said lignocellulosic biomass and generate raw pulp, wherein said digestion medium comprises alkanolamine and water having an alkanolamine to water weight ratio ranging from 60:40 to 30:70; removing the dissolved lignin from the raw pulp; and separating the raw pulp from a waste digester liquor by solid/liquid separation.

### SUMMARY OF THE INVENTION

The present invention provides for a method to produce a sugar compound from a biomass, the method comprising: (a) providing a first mixture comprising a solubilized biomass and an alkanolamine, and (b) recovering at least part of the alkanolamine from the first mixture in order to separate the at least part of the alkanolamine from the first mixture.

In some embodiments, the method further comprises: (c) introducing an enzyme and/or a microbe to the first mixture such that the enzyme and/or microbe produce a sugar from the solubilized biomass.

In some embodiments, the method further comprises: (d) the sugar is separated from the first mixture.

In some embodiments, the providing step (a) comprises incubating the first mixture at about 100° C. to about 160° C. for at least about 30 minutes.

In some embodiments, the recovering step (b) comprises distilling the at least part of the alkanolamine from the first mixture.

In some embodiments, the method further comprises: (e) introducing at least part of the alkanolamine separated in the (b) recovering step to the first mixture in step (a).

In some embodiments, the method further comprises: (f) introducing more biomass to the first mixture in step (a).

The alkanolamine is any straight or branched chain alkane comprising one or more hydroxyl and one or more amino functional groups. The amino group can be primary, secondary, or tertiay amine. The alkanolamine can be saturated or unsaturated. In some embodiments, the alkanolamine has the following structure:

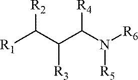

wherein R₁ to R₆ are each independently —H, —NH₂, alkyl, alkenyl, alkynyl, aryl, alkyl amine, alkenyl amine, alkynyl amine, or aryl amine, and R₁ to R₄ are each independently —OH, alkanol, alkenol, alkynol, or aryl alkanol, wherein at least one of R₁ to R₄ is —OH, alkanol, alkenol, alkynol, or aryl alkanol.

In some embodiments, the alkanolamine is an ethanolamine, aminomethyl propanol, heptaminol, propanolamine, sphingosine, methanolamine, dimethylethanolamine, or N-methylethanolamine. In some embodiments, the alkanolamine comprises 1, 2, 3, 4, 5. 6, 7, 8, 9, or 10 carbon atoms. In some embodiments, the alkanolamine comprises at least 3 carbon atoms. In some embodiments, the alkanolamine comprises 1, 2, or 3 hydroxyl functional groups. In some embodiments, the alkanolamine is a 2-aminoethan-1-ol, 1-amino-2-propanol, 2-(methylamino)ethanol, N,N-dimethylethanolamine, 1,3-diamino-2-propanol, or 2-amino-1,3-propanediol. In some embodiments, the alkanolamine is distillable.

The present invention provides for a first mixture comprising a biomass and an alkanolamine comprising at least 3 carbon atoms.

The present invention provides for a first mixture comprising a biomass and an alkanolamine having a ratio of more 1:19 by volume or weight. In some embodiments, the ratio is equal or more than about 1:18, 1:17, 1:16, 1:15, 1:14, 1:13, 1:12, or 1:11 by volume or weight.In some embodiments, the ratio is equal or more than about 1:10, 1:9, 1:8. 1:7, 1:6, 1:5, 1:4, 1:3, 1:2, or 1:1 by volume or weight.

In some embodiments, the method further comprises (c) introducing an enzyme and/or a microbe to the first mixture such that the enzyme and/or microbe produce a sugar from the solubilized biomass.

In some embodiments, the method further comprises (d) separating the sugar from the first mixture.

In some embodiments, the method results in a yield of equal to or more than about 80%, 85%, 90%, or 95% of sugar from the biomass. In some embodiments, the method results in a yield of equal to or more than about 10%, 15%, 20%, 25%, or 30% of sugar from the biomass when compared to the sugar yield obtained from the same method except alkanolamine is not present in the first mixture.

In some embodiments, step (a) does not comprise, or lacks, introducing or adding any water to the biomass or mixture. In some embodiments, the amount of water in the mixture, excluding or including water or moisture naturally found in the biomass is no more than about 10%, 9%, 8%, 7%. 6%, 5%, 4%, 3%, 2%, 1%, 0.5%, or 0.1% by weight or volume of the mixture.

The present invention provides for compositions and methods described herein. In some embodiments, the compositions and methods further comprise steps, features, and/or elements described in U.S. Pat. Application Ser. No. 16/737.724. hereby incorporated by reference in its entirety.

In some embodiments, the method, or one-pot method, does not require any solid-liquid separation step. In some embodiments, the one-pot method does not require adjustment of the pH level in the one-pot composition. In some embodiments, the one-pot method does not require any dilution, or addition of water or medium, after pretreatment and/or before saccharification and fermentation. In some embodiments, the reaction of the enzyme and the growth of the microbe occur in the same one-pot composition. In some embodiments, the method further comprises adding to or introducing to or mixing into the first mixture an IL, DES, or mixture thereof. In some embodiments, the first mixture further comprises an IL, DES, or mixture thereof.

In some embodiments, the alkanolamine, and optionally IL, DES, or mixture thereof, is renewable as it can be continuous in use. In some embodiments, the one-pot method can produce a yield of sugar that is equal to or more than about 50%, 60%, 70%. 75%, or 80%, or any other value described herein.

In some embodiments, using bio-compatible solvents enables a one-pot biomass conversion which eliminates the needs of mass transfer between reactors and the separation of solid and liquid. In some embodiments, the method does not require recycling any catalyst and/or enzyme. In some embodiments, the method requires less water usage than current biomass pretreatment. The method can produce fuels/chemicals at a higher titer and/or yield in a single vessel without any need for intermediate units of mass transfer and/or solid/liquid separation.

The present invention provides for a unique approach to biomass pretreatment involving the use of alkanolamines for the deconstruction of lignocellulosic Alkanolamines are organic bases with dual chemical functionality. Therefore, they can function as both Bronsted bases and hydrogen bond donors for effective lignin removal. Depending on the specific alkanolamine being utilized, desired physical properties such as low viscosity, low to medium boiling point can also be leveraged to enable the use of environmentally benign conditions. Preliminary results show that ethanolamine is capable of effectively pretreating biomass to release >90% sugars at a rate that is >25% more than the sugars released using the analogous ionic liquid (ethanolamine acetate). Additionally, the ethanolamine can be easily recovered at a >95% recovery rate using vacuum distillation. This approach enables a cost-effective production of fermentable sugars and lignin—a major hurdle for producing commercially viable bioenergy from waste biomass.

This invention disclosure describes a unique approach to biomass pretreatment involving the use of organic bases called “alkanolamines” for the deconstruction of the different kinds of biomass into fermentable sugars and lignin. Alkanolamines are organic bases, which contain both the amine and alcohol functionality on a simple (for example, 2-3 carbon) hydrocarbon backbone. An exemplary compound with this functional group is called hydroxyethylamine (also known as ethanolamine), however, analogous compounds such as 1-amino-2-propanol (isopropanolamine), dimethylethanolamine (2-(dimethylamino)ethanol, methylethanolamine (2-methylaminoethanol) are also suitable for this process.

Depending on the number of carbons on the backbone, and/or the isomeric conformation adopted, several key properties can be leveraged related to their performance as effective pretreatment solvents:

1. The ability to function as a Bronsted base.

2. The ability to function as a Hydrogen bond donor.

3. Low to medium boiling point for easy recovery via distillation.

4. Low viscosity.

In some embodiments, these alkanolamine compounds, or a mixture thereof, form a single component for lignin dissolution or biomass pretreatment. In these embodiments, beside the alkanolamine, no other IL or DES component is used. In past reports, certain alkanolamine compound(s) have served as components in ionic liquids (ILs) and deep eutectic solvents (DESs). However, these systems did not achieve the same effectiveness as embodiments of the present invention, which is likely due to changes in their molecular structure and their physical properties when the alkanolamines are combined with other components.

Compared to the prior art IL/DES based processes, some embodiments of the present invention have one or more of the following advantages:

Alkanolamines are much cheaper than ILs/DESs. There is no need for IL/DES synthesis and the compound is added directly into the pretreatment vessel.

Increased lignin extraction and subsequent sugar release.

Alkanomaines can be distilled at lower temperatures and be fully recovered for reuse.

Preliminary results show that ethanolamine is capable of effectively pretreating biomass (2 mm sorghum. 140° C., 3 h, 15% solid loading) in order to release >90% sugars (using 20 mg/g Ctec3/Htec3). The resultant yield is equal to or more than 25% more than the sugars released using the analogous IL (ethanolamine acetate) under the same conditions. Lastly, ethanolamine can be easily recovered at a >95% recovery rate using vacuum distillation (100° C., 1 mtorr). This enables easy recycling of the ethanolamine, as well as a one-pot saccharification/fermentation approach on the residual biomass.

The invention described herein provides one or more of the following advantages: (1) use of cheaper solvents for biomass pretreatment, (2) effective at pretreatment (via lignin extraction and reducing biomass recalcitrance), (3) facile recycling and recovery via vacuum distillation, and (4) integrated approach for the conversion of biomass to biobased fuels.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1A. Percent yield of different sugars using different compounds for pretreatment.

FIG. 1B. Chemical Structures of alkanolamines utilized in Example 2.

FIG. 2. Lignin removal and solid recovery after biomass pretreatment with alkanolamines.

FIG. 3. Glucose and xylose yields recovered after enzymatic hydrolysis of the sorghum recovered after pretreatment with alkanolamines.

FIG. 4. Lignin removal and solid recovery after biomass pretreatment with 2-aminoethan-1-ol.

FIG. 5. Glucose and xylose yields recovered after enzymatic hydrolysis of pretreated biomass with 2-aminoethan-1-ol.

FIG. 6. Glucose and xylose yields recovered after enzymatic hydrolysis of pretreated sorghum biomass with 2-aminoethan-1-ol and varying amounts of water.

FIG. 7. (A) Images of sorghum biomass changes during pretreatment and the residual lignin after the one-pot process, (B) PXRD diffractograms for the untreated and 2-aminoethan-1-ol-treated sorghum; and (C) Thermal degradation behavior of untreated and treated sorghum fibers using TGA analyses.

FIG. 8. FTIR spectra of sorghum before and after 2-aminoethan-1-ol-based pretreatment (A) in the fingerprint region (600-1,800 cm⁻¹) and (B) the region (2000-4000 cm⁻¹).

FIG. 9. Lignin monomeric composition in lignin extract analyzed by 2D ¹³C-¹H HSQC NMR spectroscopy showing the aromatic region (~6.0-8.0 /100-150 ppm). Lignin monomer ratios including tricin (T) are provided on the figures. S: syringyl, G: guaiacyl, H: p-hydroxyphenyl, pCA: p-coumarate, FA: ferulate.

FIG. 10. Process flow diagram for the one-pot conversion of sorghum biomass into biofuels using 2-aminoethan-1-ol as a pretreatment solvent.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” when applied to a value, describes a value that includes up to 10% more than the value described, and up to 10% less than the value described.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

In some embodiments, the providing step (a) comprises contacting a biomass and an alkanolamine and optionally an ionic liquid or DES. In some embodiments, the contacting step comprises introducing, adding and/or mixing the biomass with the alkanolamine and optionally the ionic liquid or DES. or vice versa.

In some embodiments, the biomass is solubilized using the alkanolamine and optionally at least part of the solvent is removed from the solubilized by separation (or washing). In some embodiments, the biomass and the alkanolamine are loaded into a vessel and homogenized. In some embodiments, the loading is solid loading and controlled at about 5%, 10%, 15%, 20%, 25%, 30%, 35%, or 40%, or a range within any two preceding values. In some embodiments, the biomass and alkanolamine are heated, such as to 100° C., 110° C., 120° C., 130° C., 140° C., 150° C., 160° C., 170° C., 180° C., 190° C., 200° C., 200° C., 212° C., or a range within any two preceding values, for a period of time, such as about 1 h, 2 h, 3 h, 4 h, or 5 h, or a range within any two preceding values. In some embodiments, after pretreatment, the mixture is cooled, such as for a period of about at least 30 mins, such as at room temperature, or about 25° C., and/or then washed at least about 1 X, 2X, 3 X, 4 X, or 5 X with water, such as deionized water. In some embodiments, the resulting solid is recovered, such as separating the solid portion with the liquid portion.

In some embodiments, the biomass is a lignocellulosic biomass. In some embodiments, the vessel is made of a material that is inert, such as stainless steel or glass, that does not react or interfere with the reactions in the pretreatment mixture.

In some embodiments, the method uses a one-pot methodology, for example, using method steps and compositions as taught in U.S. Pat. Application Ser. No. 16/737,724 (which is incorporated by reference). In some embodiments, the method further comprises heating the one-pot composition, optionally also comprising the enzyme and/or microbe, to a temperature that is equal to, about, or near the optimum temperature for the enzymatic activity of the enzyme and/or growth of the microbe. In some embodiments, the enzyme is a genetically modified host cell capable of converting the cellulose in the biomass into a sugar. In some embodiments, there is a plurality of enzymes. In some embodiments, the microbe is a genetically modified host cell capable of converting a sugar produced from the biomass into a biofuel and/or chemical compound. In some embodiments, there is a plurality of microbes. In some embodiments, the method produces a sugar and a lignin from the biomass. The lignin can further be processed to produce a DES. The sugar is used for growth by the microbe.

In some embodiments, the solubilizing is full, near full (such as at least about 70, 80, or 90%), or partial (such as at least about 10, 20, 30, 40, 50. or 60%). In some embodiments, the one-pot composition is a slurry. When the steps (a) and (b), and optionally steps (c) and/or (d), are continuous, the one-pot composition is in a steady state.

### Ionic Liquid

Ionic liquids (ILs) are salts that are liquids rather than crystals at room temperatures. It will be readily apparent to those of skill that numerous ILs can be used in the present invention. In some embodiments of the invention, the IL is suitable for pretreatment of the biomass and for the hydrolysis of cellulose by thermostable cellulase. Suitable ILs are taught in ChcmFiles (2006) 6(9) (which are commercially available from Sigma-Aldrich, Milwaukee, Wis.). Such suitable ILs include, but are not limited to, 1-alkyl-3-alkylimidazolium alkanate, 1-alkyl-3-alkylimidazolium alkylsulfate, 1-alkyl-3-alkylimidazolium methylsulfonate, 1-alkyl-3-alkylimidazolium hydrogensulfate, 1-alkyl-3-alkylimidazolium thiocyanate, and 1-alkyl-3-alkylimidazolium halide, wherein an “alkyl” is an alkyl group comprising from 1 to 10 carbon atoms, and an “alkanate” is an alkanate comprising from 1 to 10 carbon atoms. In some embodiments, the “alkyl” is an alkyl group comprising from 1 to 4 carbon atoms. In some embodiments, the “alkyl” is a methyl group, ethyl group or butyl group. In some embodiments, the “alkanate” is an alkanate comprising from 1 to 4 carbon atoms. In some embodiments, the “alkanate” is an acetate. In some embodiments, the halide is chloride.

In some embodiments, the IL includes, but is not limited to, 1-ethyl-3-methylimidazolium acetate (EMIN Acetate), 1-ethyl-3-methylimidazolium chloride (EMIN Cl), 1-ethyl-3-methylimidazolium hydrogensulfate (EMIM HOSO₃), 1-ethyl-3-methylimidazolium methylsulfate (EMIM MeOSO₃), 1-ethyl-3-methylimidazolium ethylsulfate (EMIM EtOSO₃), 1-ethyl-3-methylimidazolium methanesulfonate (EMIM MeSO₃), 1-ethyl-3-methylimidazolium tetrachloroaluminate (EMIM AlCl₄), 1-ethyl-3-methylimidazolium thiocyanate (EMIM SCN). 1-butyl-3-methylimidazolium acetate (BMIM Acetate), 1-butyl-3-methylimidazolium chloride (BMIM Cl), 1-butyl-3-methylimidazolium hydrogensulfate (BMIM HOSO₃), 1-butyl-3-methylimidazolium methanesulfonate (BMIM MeSO₃), 1-butyl-3-methylimidazolium methylsulfate (BMIM MeOSO₃), 1-butyl-3-methylimidazolium tetrachloroaluminate (BMIM AlCl4), 1-butyl-3-methylimidazolium thiocyanate (BMIM SCN), 1-ethyl-2,3-dimethylimidazolium ethylsulfate (EDIM EtOSO₃), Tris(2-hydroxyethyl)methylammonium methylsulfate (MTEOA MeOSO₃), 1-methylimidazolium chloride (MIM Cl), 1-methylimidazolium hydrogensulfate (MIM HOSO₃), 1,2,4-trimethylpyrazolium methylsulfate, tributylmethylammonium methylsulfate, choline acetate, choline salicylate, and the like.

In some embodiments, the ionic liquid is a chloride ionic liquid. In other embodiments, the ionic liquid is an imidazolium salt. In still other embodiments, the ionic liquid is a 1-alkyl-3-imidazolium chloride, such as 1-ethyl-3-methylimidazolium chloride or 1-butyl-3-methylimidazolium chloride.

In some embodiments, the ionic liquids used in the invention are pyridinium salts, pyridazinium salts, pyrimidium salts, pyrazinium salts, imidazolium salts, pyrazolium salts, oxazolium salts. 1,2.3-triazolium salts, 1,2,4-triazolium salts, thiazolium salts, isoquinolium salts, quinolinium salts isoquinolinium salts, piperidinium salts and pyrrolidinium salts. Exemplary anions of the ionic liquid include, but are not limited to halogens (e.g., chloride, floride, bromide and iodide), pseudohalogens (e.g., azide and isocyanate), alkyl carboxylate, sulfonate, acetate and alkyl phosphate.

Additional ILs suitable for use in the present invention are described in U.S. Pat. Nos. 6,177,575; 9,765,044; and, 10,155,735; U.S. Pat. Application Publication Nos. 2004/0097755 and 2010/0196967; and, PCT International Pat. Application Nos. PCT/US2015/058472, PCT/US2016/063694, PCT/US2017/067737, and PCT/US2017/036438 (all of which are incorporated in their entireties by reference). It will be appreciated by those of skill in the art that others ILs that will be useful in the process of the present invention are currently being developed or will be developed in the future, and the present invention contemplates their future use. The ionic liquid can comprise one or a mixture of the compounds.

In some embodiments, the IL is a protic ionic liquid (PIL). Suitable protic ionic liquids (PILs) include fused salts with a melting point less than 100° C. with salts that have higher melting points referred to as molten salts. Suitable PPILs are disclosed in Greaves et al. “Protic Ionic Liquids: Properties and Applications” Chem. Rev. 108(1):206-237 (2008). PILs can be prepared by the neutralization reaction of certain Brønsted acids and Brønsted bases (generally from primary, secondary or tertiary amines, which are alkaline) and the fundamental feature of these kinds of ILs is that their cations have at least one available proton to form hydrogen bond with anions. In some embodiments, the protic ionic liquids (PILs) are formed from the combination of organic ammonium-based cations and organic carboxylic acid-based anions. PILs are acid-base conjugate ILs that can be synthesized via the direct addition of their acid and base precursors. In some embodiments, the PIL is a hydroxyalkylammonium carboxylate. In some embodiments, the hydroxyalkylammonium comprises a straight or branched C1, C2, C3, C4, C5, C6, C7, C8, C9, or C10 chain. In some embodiments, the carboxylate comprises a straight or branched C1, C2, C3, C4, C5, C6, C7. C8, C9, or C10 chain. In some embodiments, the carboxylate is substituted with one or more hydroxyl groups. In some embodiments, the PIL is a hydroxyethylammonium acetate.

In some embodiments, the protic ionic liquid (PIL) is disclosed by U.S. Pat. Application Publication No. 2004/0097755, hereby incorporated by reference.

Suitable salts for the method include combinations of organic ammonium-based cations (such as ammonium, hydroxyalkylammonium, or dimethylalkylammonium) with organic carboxylic acid-based anions (such as acetic acid derivatives (C1-C8), lactic acid, glycolic acid, and DESs such as ammonium acetate/lactic acid).

Suitable IL, such as distillable IL, are disclosed in Chen et al. “Distillable Ionic Liquids: reversible Amide O Alkylation”, Angewandte Comm. 52:13392-13396 (2013), King et al. “Distillable Acid-Base Conjugate Ionic Liquids for Cellulose Dissolution and Processing”, Angewandte Comm. 50:6301-6305 (2011), and Vijayaraghavan et al. “CO₂-based Alkyl Carbamate Ionic Liquids as Distillable Extraction Solvents”, ACS Sustainable Chem. Engin. 2:31724-1728 (2014), all of which are hereby incorporated by reference.

Suitable PIL, such as distillable PIL, are disclosed in Idris et al. “Distillable Protic Ionic Liquids for Keratin Dissolution and Recovery”, ACS Sustainable Chem. Engin. 2:1888-1894 (2014) and Sun et al. “One-pot integrated biofuel production using low-cost biocompatible protic ionic liquids”. Green Chem. 19(13:):3152-3163 (2017), all of which are hereby incorporated by reference.

In some embodiments, the PILs) are formed with the combination of organic ammonium-based cations and organic carboxylic acid-based anions. PILs are acid-base conjugate ILs that can be synthesized via the direct addition of their acid and base precursors. Additionally, when sufficient energy is employed, they can dissociate back into their neutral acid and base precursors, while the PILs are re-formed upon cooling. This presents a suitable way to recover and recycle the ILs after their application. In some embodiments, the PIL (such as hydroxyethylammonium acetate - [Eth][OAc]) is an effective solvent for biomass pretreatment and is also relatively cheap due to its ease of synthesis (Sun et al., Green Chem. 19(13):3152-3163 (2017)).

### Deep Eutectic Solvent (DES)

DESs are systems formed from a eutectic mixture of Lewis or Brønsted acids and bases which can contain a variety of anionic and/or cationic species. DESs can form a eutectic point in a two-component phase system. DESs are formed by complexation of quaternary ammonium salts (such as, choline chloride) with hydrogen bond donors (HBD) such as amines, amides, alcohols, or carboxylic acids. The interaction of the HBD with the quaternary salt reduces the anion-cation electrostatic force, thus decreasing the melting point of the mixture. DESs share many features of conventional ionic liquid (IL), and promising applications would be in biomass processing, electrochemistry, and the like. In some embodiments, the DES is any combination of Lewis or Brønsted acid and base. In some embodiments, the Lewis or Brønsted acid and base combination used is distillable.

In some embodiments, DES is prepared using an alcohol (such as glycerol or ethylene glycol), amines (such as urea), and an acid (such as oxalic acid or lactic acid). The present invention can use renewable DESs with lignin-derived phenols as HBDs. Both phenolic monomers and phenol mixture readily form DES upon heating at 100° C. with specific molar ratio with choline chloride. This class of DES does not require a multistep synthesis. The DES is synthesized from lignin which is a renewable source.

Both monomeric phenols and phenol mixture can be used to prepare DES. DES is capable of dissolving biomass or lignin, and can be utilized in biomass pretreatment and other applications. Using DES produced from biomass could lower the cost of biomass processing and enable greener routes for a variety of industrially relevant processes.

The DES. or mixture thereof, is bio-compatible: meaning the DES, or mixture thereof, does not reduce or does not significantly reduce the enzymatic activity of the enzyme, and/or is not toxic, and/or does not reduce or significantly reduce, the growth of the microbe. A “significant” reduction is a reduction to 70, 80, 90, or 95% or less of the enzyme’s enzymatic activity and/or the microbe’s growth (or doubling time), if the DES, or mixture thereof, was not present.

In some embodiments, the DES, or mixture thereof, comprises a quaternary ammonium salt and/or glycerol. In some embodiments, the DES, or mixture thereof, comprises a quaternary ammonium salt and/or glycerol. In some embodiments, the quaternary ammonium salt and/or glycerol have a molar ratio of about 1:1 to about 1:3. In some embodiments, the quaternary ammonium salt and/or glycerol have a molar ratio of about 1:1.5 to about 1:2.5. In some embodiments, the quaternary ammonium salt and/or glycerol have a molar ratio of about 1:1.8 or 1:1.9 to about 1:2.1 or 1:2.2. In some embodiments, the quaternary ammonium salt and/or glycerol have a molar ratio of about 1:2. In some embodiments, the quaternary ammonium salt is a choline halide, such choline chloride.

In some embodiments, the DES is distillable if the DABCS or DES can be recovered at least equal to or more than about 50%, 55%, 60%, 65%, 70%, 75%, 80%, or 85% yield by distilling over vacuum at a temperature at about 100° C., 110° C., 120° C., 130° C., 140° C., 150° C., or 160° C., or any temperature between any two of the preceding temperatures.

In some embodiments, the DES can be one taught in WO 2018/204424 (Seema Singh et al.), which is hereby incorporated in its entirety by reference.

In some embodiments, the method further comprises heating the one-pot composition, optionally also comprising the enzyme and/or microbe, to a temperature that is equal to, about, or near the optimum temperature for the enzymatic activity of the enzyme and/or growth of the microbe. In some embodiments, the enzyme is a genetically modified host cell capable of converting the cellulose in the biomass into a sugar. In some embodiments, there is a plurality of enzymes. In some embodiments, the microbe is a genetically modified host cell capable of converting a sugar produced from the biomass into a biofuel and/or chemical compound. In some embodiments, there is a plurality of microbes. In some embodiments, the introducing steps (a) and (b) together produce a sugar and a lignin from the biomass. The lignin can further be processed to produce a DES. The sugar is used for growth by the microbe.

In some embodiments, the solubilizing is full, near full (such as at least about 70, 80, or 90%), or partial (such as at least about 10, 20, 30, 40, 50, or 60%). In some embodiments, the one-pot composition is a slurry. When the steps (a) to (c) are continuous, the one-pot composition is in a steady state.

In some embodiments, all or some of the one-pot composition is further pretreated as follows: the method further comprising: (d) optionally separating the sugar and the lignin in the one-pot composition, (e) depolymerizing and/or converting the lignin into one or more lignin derived monomeric phenol, or a mixture thereof, (f) providing the one or more lignin derived monomeric phenol, or a mixture thereof, in a solution, (g) introducing one or more quaternary ammonium salts, or a mixture thereof, to the solution, (h) heating the solution, such that steps (g) and (h) together result in the synthesis of a DES, (i) optionally forming a DES system from the DES synthesized in step (h), and (j) optionally repeating steps (d) to (i) using the DES system formed in step (i) in the introducing step (a).

In some embodiments, the heating step (h) comprises increasing the temperature of the solution to a value within a range of about 75° C. to about 125° C. In some embodiments, the heating step (h) comprises increasing the temperature of the solution to a value within a range of about 80° C. to about 120° C. In some embodiments, the heating step (h) comprises increasing the temperature of the solution to a value within a range of about 90° C. to about 110° C. In some embodiments, the heating step (h) comprises increasing the temperature of the solution to about 100° C.

### Enzyme

In some embodiments, the enzyme is a cellulase. In some embodiments, the enzyme is thermophilic or hyperthermophilic. In some embodiments, the enzyme is any enzyme taught in U.S. Pat. Nos. 9,322,042; 9,376,728; 9,624,482; 9,725,749; 9,803,182; and 9,862,982; and PCT International Patent Application Nos. PCT/US2015/000320, PCT/US2016/063198, PCT/US2017/036438, PCT/US2010/032320, and PCT/US2012/036007 (all of which are incorporated in their entireties by reference).

### Microbe

In some embodiments, the microbe is any prokaryotic or eukaryotic cell, with any genetic modifications, taught in U.S. Pat. Nos. 7,985,567; 8,420,833; 8,852,902; 9,109,175; 9.200.298; 9,334,514; 9,376,691; 9,382,553; 9,631,210; 9,951,345; and 10,167,488; and PCT International Patent Application Nos. PCT/US14/48293, PCT/US2018/049609, PCT/US2017/036168, PCT/US2018/029668, PCT/US2008/068833, PCT/US2008/068756, PCT/US2008/068831. PCT/US2009/042132, PCT/US2010/033299, PCT/US2011/053787, PCT/US2011/058660, PCT/US2011/059784. PCT/US2011/061900, PCT/US2012/031025, and PCT/US2013/074214 (all of which are incorporated in their entireties by reference).

Generally, although not necessarily, the microbe is a yeast or a bacterium. In some embodiments, the microbe is Rhodosporidium toruloides or Pseudomonas putida. In some embodiments, the microbe is a Gram negative bacterium. In some embodiments, the microbe is of the phylum Proteobactera. In some embodiments, the microbe is of the class Gammaproteobacteria. In some embodiments, the microbe is of the order Enterobacteriales. In some embodiments, the microbe is of the family Enterobacteriaceae. Examples of suitable bacteria include, without limitation, those species assigned to the Escherichia, Enterobacter, Azotobacter, Erwinia, Bacillus, Pseudomonas, Klebsielia, Proteus, Salmonella, Serratia, Shigella, Rhizobia. Vitreoscilla, and Paracoccus taxonomical classes. Suitable eukaryotic microbes include, but are not limited to, fungal cells. Suitable fungal cells are yeast cells, such as yeast cells of the Saccharomyces genus.

Yeasts suitable for the invention include, but are not limited to, Yarrowia, Candida, Bebaromyces, Saccharomyces, Schizosaccharomyces and Pichia cells. In some embodiments, the yeast is Saccharomyces cerevisae. In some embodiments, the yeast is a species of Candida, including but not limited to C. tropicalis, C. maltosa, C. apicola, C. paratropicalis, C. albicans, C. cloacae. C. guillermondii, C. intermedia, C. lipolytica, C. panapsilosis and C. zeylenoides. In some embodiments, the yeast is Candida tropicalis. In some embodiments, the yeast is a non-oleaginous yeast. In some embodiments, the non-oleaginous yeast is a Saccharomyces species. In some embodiments, the Saccharomyces species is Saccharomyces cerevisiae. In some embodiments, the yeast is an oleaginous yeast. In some embodiments, the oleaginous yeast is a species. In some embodiments, the Rhodosporidium species is Rhodosporidium toruloides.

In some embodiments the microbe is a bacterium. Bacterial host cells suitable for the invention include, but are not limited to, Escherichia, Corynebacterium, Pseudomonas, Streptomyces, and Bacillus. In some embodiments, the Escherichia cell is an E. coli, E. albertii, E. fergusonii, E. hermanii, E. marmotae, or E. vulneris. In some embodiments, the Corynebacterium cell is Corynebacterium glutamicum, Corynebacterium kroppenstedtii, Corynebacterium alimapuense, Corynebacterium amycolatum, Corynebacterium diphtheriae, Corynebacterium efficiens, Corynebacterium jeikeium. Corynebacterium macginleyi, Corynebacterium matruchotii, Corynebacterium minutissimum, Corynebacterium renale, Corynebacterium striatum, Corynebacterium ulcerans, Corynebacterium urealyticum, or Corynebacterium uropygiale. In some embodiments, the Pseudomonas cell is a P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P. oryzihabitans, P. parafluva, or P. plecoglossicida. In some embodiments, the Streptomyces cell is a S. coelicolor, S. lividans, S. venezuelae, S. ambofaciens, S. avermitilis, S. albus, or S. scabies. In some embodiments, the Bacillus cell is a B. subtilis, B. megaterium, B. licheniformis, B. anthracis, B. amyloliquefaciens, or B. pumilus.

### Biofuel

In some embodiments, the biofuel produced is ethanol, or any other organic molecule, described produced in a cell taught in U.S. Pat. Nos. 7,985,567; 8,420,833; 8,852.902; 9,109,175; 9,200,298; 9,334,514; 9,376,691; 9,382,553; 9,631,210; 9,951,345; and 10,167,488; and PCT International Patent Application Nos. PCT/US14/48293, PCT/US2018/049609, PCT/US2017/036168, PCT/US2018/029668, PCT/US2008/068833, PCT/US2008/068756, PCT/US2008/068831, PCT/US2009/042132, PCT/US2010/033299. PCT/US2011/053787, PCT/US2011/058660, PCT/US2011/059784, PCT/US2011/061900, PCT/US2012/031025, and PCT/US2013/074214 (all of which are incorporated in their entireties by reference).

### Biomass

The biomass comprising the lignin can be any biomass disclosed herein. The biomass can be obtained from one or more feedstock, such as softwood feedstock, hardwood feedstock, grass feedstock, and/or agricultural feedstock, or a mixture thereof. In some embodiments, the biomass is a lignocellulosic biomass comprising cellulose, hemicellulose, and lignin in various ratios (depending on the biomass source). The cellulose, hemicellulose, and lignin are held together by covalent and strong hydrogen bonds forming a complex matrix recalcitrant to facile depolymerization. The biomass can also be from any post-production or post-consumer source that comprises lignin and/or lignosulfonate, such as used coffee grounds, spent pulping liquids (red or brown liquor) from sulfite pulping, or a wastestream.

Softwood feedstocks include, but are not limited to, Araucaria (e.g. A. cunninghamii, A. angustifolia, A. araucana); softwood Cedar (e.g. Juniperus virginiana, Thuja plicata, Thuja occidentalis. Chamaecyparis thyoides Callitropsis nootkatensis); Cypress (e.g. Chamaecyparis, Cupressus Taxodium. Cupressus arizonica, Taxodium distichum. Chamaecyparis obtusa, Chamaecyparis lawsoniana. Cupressus semperviren); Rocky Mountain Douglas fir; European Yew; Fir (e.g. Abies balsamea, Abies alba, Abies procera, Abies amabilis); Hemlock (e.g. Tsuga canadensis, Tsuga mertensiana. Tsuga heterophylla); Kauri; Kaya; Larch (e.g. Larix decidua, Larix kaempferi, Larix laricina, Larix occidentalis); Pine (e.g. Pinus nigra, Pinus banksiana, Pinus contorta, Pinus radiata. Pinus ponderosa, Pinus resinosa, Pinus sylvestris. Pinus strobus, Pinus monticola, Pinus lambertiana, Pinus taeda, Pinus palustris, Pinus rigida, Pinus echinata); Redwood; Rimu; Spruce (e.g. Picea abies, Picea mariana, Picea rubens, Picea sitchensis, Picea glauca); Sugi; and combinations/hybrids thereof.

For example, softwood feedstocks which may be used herein include cedar; fir; pine; spruce; and combinations thereof. The softwood feedstocks for the present invention may be selected from loblolly pine (Pinus taeda), radiata pine, jack pine, spruce (e.g., white, interior, black), Douglas fir. Pinus silvestris, Picea abies, and combinations/hybrids thereof. The softwood feedstocks for the present invention may be selected from pine (e.g. Pinus radiata, Pinus taeda); spruce; and combinations/hybrids thereof.

Hardwood feedstocks include, but are not limited to, Acacia; Afzelia; Synsepalum duloificum; Albizia ; Alder (e.g. Alnus glutinosa, Alnus rubra ); Applewood; Arbutus ; Ash (e.g. F. nigra, F. quadrangulata, F. excelsior, F. pennsylvanica lanceolata, F. latifolia, F. profunda, F. americana ); Aspen (e.g. P. grandidentata, P. tremula, P. tremuloides ); Australian Red Cedar ( Toona ciliata ); Ayna ( Distemonanthus benthamianus ); Balsa ( Ochroma pyramidale ); Basswood (e.g. T. americana, T. heterophylla ); Beech (e.g. F. sylvatica, F. grandifolia ); Birch; (e.g. Betula populifolia, B. nigra, B. papyrifera, B. lenta, B. alleghaniensis/ B. lutea, B. pendula, B. pubescens ); Blackbean; Blackwood; Bocote; Boxelder; Boxwood; Brazilwood; Bubing a; Buckeye (e.g. Aesculus hippocastanum. Aesculus glabra, Aesculus flava/Aesculus octandra ); Butternut; Catalpa; Chemy (e.g. Prunus serotina, Prunus pennsylvanica, Prunus avium ); Crabwood; Chestnut; Coachwood; Cocobolo; Corkwood; Cottonwood (e.g. Populus balsamifera, Populus deltoides, Populus sargentii, Populus heterophylla ); Cucumbertree; Dogwood (e.g. Comus florida, Comus nuttallii ); Ebony (e.g. Diospyros kurzii, Diospyros melanida, Diospyros crassiflora ); Elm (e.g. Ulmus americana. Ulmus procera, Ulmus thomasii, Ulmus rubra, Ulmus glabra); Eucalyptus ; Greenheart; Grenadilla; Gum (e.g. Nyssa sylvatica, Eucalyptus globulus, Liquidambar styraciflua, Nyssa aquatica ); Hickory (e.g. Carya alba, Carya glabra, Carya ovata, Carya laciniosa ); Hornbeam; Hophornbeam; Ipê; Iroko; Ironwood (e.g. Bangkirai. Carpinus caroliniana, Casuarina equisetifolia, Choricbangarpia subargentea, Copaifera spp., Eusideroxylon zwageri, Guajacum officinale, Guajacum sanctum, Hopea odorata, Ipe, Krugiodendronferreum, Lyonothamnus lyonii ( L. floribundus), Mesua ferrea, Olea spp., Olneya tesota, Ostrya virginiana, Parrotia persica, Tabebuia serratifolia ); Jacarandá; Jotoba; Lacewood; Laurel; Limba; Lignum vitae; Locust (e.g. Robinia pseudacacia, Gleditsia triacanthos ); Mahogany; Maple (e.g. Acer saccharum, Acer nigrum, Acer negundo, Acer rubrum, Acer saccharinum. Acer pseudoplatanus ); Meranti; Mpingo; Oak (e.g. Quercus macrocarpa, Quercus alba. Quercus stellata, Quercus bicolor. Quercus virginiana, Quercus michauxii, Quercus prinus, Quercus muhlenbergii, Quercus chrysolepis, Quercus lyrata, Quercus robur, Quercus petraea. Quercus rubra, Quercus velutina. Quercus laurifolia, Quercus falcata. Quercus nigra. Quercus phellos. Quercus texana ); Obeche; Okoumé; Oregon Myrtle; California Bay Laurel; Pear; Poplar (e.g. P. balsamifera, P. nigra, Hybrid Poplar ( Populus×canadensis )); Ramin; Red cedar; Rosewood; Sal; Sandalwood; Sassafras; Satinwood; Silky Oak; Silver Wattle; Snakewood; Sourwood; Spanish cedar; American sycamore; Teak; Walnut (e.g. Juglans nigra, Juglans regia); Willow (e.g. Salix nigra. Salix alba); Yellow poplar ( Liriodendron tulipifera); Bamboo; Palmwood; and combinations/hybrids thereof.

For example, hardwood feedstocks for the present invention may be selected from Acacia, Aspen, Beech, Eucalyptus, Maple, Birch, Gum, Oak, Poplar, and combinations/hybrids thereof. The hardwood feedstocks for the present invention may be selected from Populus spp. (e.g. Populus tremuloides), Eucalyptus spp. (e.g. Eucalyptus globulus), Acacia spp. (e.g. Acacia dealbata), and combinations thereof.

Grass feedstocks include, but are not limited to, C₄ or C₃ grasses, e.g. Switchgrass, Indiangrass, Big Bluestem, Little Bluestem, Canada Wildrye, Virginia Wildrye, and Goldenrod wildflowers, etc, amongst other species known in the art.

Agricultural feedstocks include, but are not limited to, agricultural byproducts such as husks, stovers, foliage, and the like. Such agricultural byproducts can be derived from crops for human consumption, animal consumption, or other non-consumption purposes. Such crops can be corps such as corn, wheat, sorghum, rice, soybeans, hay, potatoes, cotton, or sugarcane. The feedstock can arise from the harvesting of crops from the following practices: intercropping, mixed intercropping, row cropping, relay cropping, and the like.

In some embodiments, the biomass is an ensiled biomass. In some embodiment, the biomass is ensiled by placing the biomass in an enclosed container or room, such as a silo, or by piling it in a heap covered by an airproof layer, such as a plastic film. The biomass undergoing the ensiling, known as the silage, goes through a bacterial fermentation process resulting in production of volatile fatty acids. In some embodiment, the ensiling comprises adding ensiling agents such as sugars, lactic acid or inculants. In some embodiments, the ensiled biomass comprises one or more toxic compounds. In some embodiments, when ensiled biomass comprises one or more toxic compounds, the microbe is resistant to the one or more toxic compounds.

### Example 1

### Materials

### Biomass

The main biomass utilized was Sorghum (Sorghum bicolor), which was donated by Idaho National Labs (Idaho Falls, ID). The biomass was dried for 24 h in a 40° C. oven. Subsequently, it was a knife-milled with a 2 mm screen (Thomas-Wiley Model 4, Swedesboro, NJ). The resulting biomass was then placed in a leak-proof bag and stored in a cool dry place. Additional biomass studied include the forest residues generated from California woody biomass such as pine, walnut, almond, fir. These feedstocks were generously donated by Aemetis, Inc. (Cupertino, CA). They were also prepared and stored using similar conditions.

### Chemicals (Alkanolamines)

The following alkanolamines were purchased from Sigma Aldrich (St. Louis, MO) and used as received: 2-aminoethan-1-ol (≥99% purity), 1-amino-2-propanol (93% purity), 2-(Methylamino)ethanol (≥98% purity). N.N-dimethylethanolamine, (≥99.5% purity), 1.3-diamino-2-propanol (96.5%), 2-amino-1,3-propanediol (98% purity).

### Enzymes

Novozymes A/S’s (Bagsvaerd. Denmark) cellulase and hemicellulase complexes Cellic® CTec3 and Htec3 were used as received.

### Methods

### Biomass Pretreatment

The biomass pretreatment was carried out using the conventional method that involves early separation (or washing) to remove the solvent after pretreatment (prior to downstream conversion). In a typical experiment, 1 g of the biomass and the solvent were loaded into an ace pressure tube (50 mL, Ace Glass Inc., Vineland, NJ) and homogenized. The solid loading was controlled at 15% and heated in an oil bath set to 140° C. for 3 h. After pretreatment, the mixture could cool for 30 mins and then washed 5 X with deionized water using a 40 mL centrifugation-decanting cycle. The recovered solid was gravimetrically tracked to determine the solid recovery, while also passing through enzymatic hydrolysis and compositional analysis (see below).

### Enzymatic Hydrolysis

For enzymatic hydrolysis, the 0.15 g of the recovered biomass was loaded into a test-tube at 1.5 wt% solids loading. The liquid fraction contained 50 vol% of a 0.1 M citrate buffer (pH 5), 1 vol% NaN₃ and 20 mg protein/g biomass using a 9/1 mixture of the CTec3/Htec3 and completed with deionized water to attain the desired solid loading. The mixture was subsequently incubated at 50° C. for 72 h in a rotary incubator (Enviro-Genie, Scientific Industries, Inc.). The amount of sugars released were quantified using HPLC after the incubation was complete.

### Compositional Analysis

Compositional analysis of the biomass before and after pretreatment was performed using an adapted NREL method. 1.5 mL of 72 wt% Sulfuric Acid was added to 0.15 grams of pretreated biomass and subsequently allowed to incubate for 60 minutes at 30° C. and 200 rpm. Next, 42 mL of deionized water was added, and the samples were autoclaved for 1 h (using liquids cycle 121 C). Samples were then filtered through crucibles and the first 10 mL of the filtered solution was saved for future analysis. Remaining biomass was washed using 25 mL of water and crucibles were placed in a 105° C. oven for drying. High Performance Liquid Chromatography (HPLC) was used to examine the glucose and xylose contents. Lignin was characterized as acid soluble (ASL) and insoluble (AIL) fractions. The ASL was determined spectroscopically using the absorbance at 240 nm, while AIL is the recovered residue after filtration (tracked gravimetrically). Acid-insoluble lignin was quantified gravimetrically from the solid after heating overnight at 105° C. (the weight of acid-insoluble lignin + ash) and then 575° C. for at least 6 h (the weight of ash).

### Results and Discussion

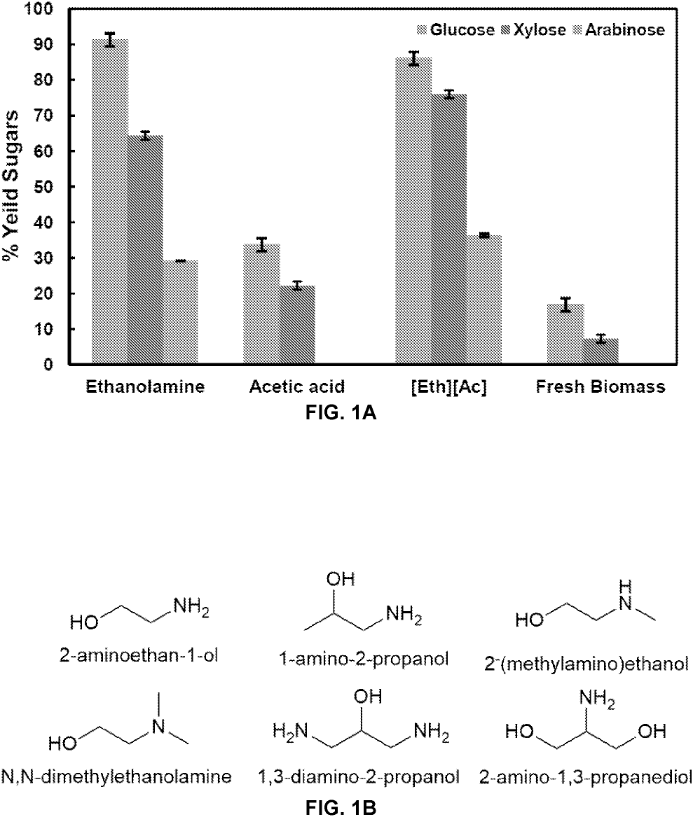

Preliminary results show that ethanolamine is capable of effectively pretreating in order to release >90% sugars. The resultant yield is more than the sugars released using the analogous IL (ethanolamine acetate) under the same conditions (FIG. 1A).

### Example 2

### Materials

The main biomass utilized was Sorghum (Sorghum bicolor), which was donated by Idaho National Labs (Idaho Falls, ID). The biomass was dried for 24 h in a 40° C. oven. Subsequently, it was a knife-milled with a 2 mm screen (Thomas-Wiley Model 4, Swedesboro, NJ). The resulting biomass was then placed in a leak-proof bag and stored in a cool dry place. Additional biomass studied included the forest residues generated from California woody biomass such as pine, walnut, almond, and fir. These feedstocks were generously donated by Aemetis, Inc. (Cupertino, CA). They were also prepared and stored using similar conditions (dried for 24 h in a 40° C. oven). The following alkanolamines were purchased from Sigma Aldrich (St. Louis, MO) and used as received: 2-aminoethan-1-ol (≥99% purity), 1-amino-2-propanol (93% purity). 2-(Methylamino)ethanol (≥98% purity). N.N-dimethylethanolamine, (≥99.5% purity), 1,3-diamino-2-propanol (96.5%), 2-amino-1,3-propanediol (98% purity), citric acid (ACS reagent ≥99.5%), sodium citrate tribasic dihydrate (ACS reagent, ≥99.0%) and sodium azide. Sulfuric acid (72% and 95-98%) was purchased from VWR), and sugar standards glucose (≥99.5%), xylose (≥99%), and arabinose (≥98%) were procured from Sigma-Aldrich for high-performance liquid chromatography (HPLC) analysis. Commercial cellulase (Cellic® CTec3) and hemicellulase (Cellic® HTec3) mixtures were provided by Novozymes, North America (Franklinton, NC).

### Methods

### Biomass Pretreatment

The biomass pretreatment was carried out using the conventional method that involves early separation (or washing) to remove the solvent after pretreatment (prior to downstream conversion). In a typical experiment, 1 g of the biomass and the solvent was loaded into an ace pressure tube (50 mL, Ace Glass Inc., Vineland. NJ) and homogenized. The solid loading was controlled at 20 wt% and heated in an oil bath set to 140° C. for 3 h. After pretreatment, the mixture was allowed to cool for 30 mins and then washed 5 X with deionized water using a 40 mL centrifugation-decanting cycle. Finally, the recovered solid fraction was lyophilized and then gravimetrically tracked to determine the solid recovery (SR), while also passing through enzymatic hydrolysis (EH) and compositional analysis (CA). All the experiments were performed in duplicate, and the average values are detailed here. The solid recovery (%SR) after pretreatment was calculated based on the following equation. The selection of the initial conditions was based on previous results demonstrating pretreatment effectiveness and loosely based on the pretreatment severity factor. 14,31,56 Additionally optimization on various factors such as pretreatment time, temperature and the solid loading was conducted (see below).

$$\begin{matrix}\% \text{Solid Recovery} \left(\% \text{SR}\right) = \\ \frac{\text{Weight of biomass recovered after pretreatment}}{\text{Weight of biomass used for pretreatment}} \times 100\end{matrix}$$

### Enzymatic Hydrolysis

The enzymatic saccharification of pretreated and untreated biomass was carried out using commercially available enzymes, Cellic® Ctec3 and Htec3 (9:1 v/v) from Novozymes, at 50° C. in a rotary incubator (Enviro-Genie. Scientific Industries, Inc.). All reactions were performed at 1.5 wt% biomass loading in a 15 mL centrifuge tube (using 0.15 g of the pretreated or untreated biomass). The pH of the mixture was adjusted to 5 with 100 mM sodium citrate buffer supplemented with 0.1 wt% sodium azide to prevent microbial contamination. The total reaction volume included a total protein content of 20 mg/g biomass. The number of sugars released was analyzed on an Agilent HPLC 1260 infinity system (Santa Clara, CA) equipped with a Bio-Rad Aminex HPX-87H column (300 × 7.8 mm2) and a Refractive Index detector. An aqueous solution of sulfuric acid (4 mM) was used as the eluent (0.6 mL/min, column temperature 60° C.). All enzymatic saccharification was conducted in duplicate. The sugar yield was calculated as an overall process yield using the formula below (equation 2), which accounts for sugars/oligosaccharides lost during pretreatment/washing.

$$\begin{matrix}\% \text{Sugar Yield Process} = \\ \% \text{SR} \times \frac{\text{Weight sugars released after hydrolysis}}{\text{Weight of sugars in the original biomass}}\end{matrix}$$

### Compositional Analysis

The biomass compositional analysis of pretreated and untreated biomass sorghum was performed to determine the glucan, xylan, lignin, ash and extractive content by utilizing the two-step acid hydrolysis procedure previously described by NREL.57 Dried biomass samples were extracted sequentially using the solvents: water, 80% ethanol/water, and acetone.58 Typically, 1 g of biomass was combined to a tube containing 40 mL of the solvent of choice. The mixture was then homogenized, sonicated for 20 minutes, and then centrifuged (10 min, 4000 RPM) to separate the extracts/solvents from the residual biomass. This extraction cycle was carried out 5 times for each biomass/solvent. Finally, the residual biomass was dried overnight at 40° C. and utilized for further compositional analyses. In summary. 150 mg of the dry extractive-free biomass was exposed to 1.5 mL of 72% w/w H₂SO₄ and incubated at 30° C. for 1 hr. Subsequently, the mixture was taken through secondary hydrolysis at 4% w/w H₂SO₄ at 121° C. for 1 hr. After the two-step acid hydrolysis, the hydrolysates were filtered using medium porosity filtering crucibles. The filtrates were then spectrophotometrically analyzed for the acid-insoluble lignin (ASL) (NanoDrop 2000, Thermo Fisher Scientific, Waltham. MA) using the absorbance at 240 nm. Additionally, monomeric sugars (glucose and xylose) were determined by HPLC using an Agilent 1200 series instrument equipped with a refractive index detector and Bio-Rad Aminex HPX-87H column, coupled with a guard column assembly. Product separation was obtained at 60° C. with 4 mM H₂SO₄ as a mobile phase at a flow rate of 0.6 mL/min. Finally, the Klason lignin (acid-insoluble lignin - ASL) was determined gravimetrically by subtracting the weight of the oven-dried residual solids (105° C.) and the ash content (575° C.). All compositional analyses were conducted in duplicate. The amount of lignin removed can be calculated using the formula below (equation 3). Note: % Lignin = %AIL + %ASL.

$$\begin{matrix}\% \text{Lignin Removal} = \\ 100 - \% \text{SR} \times \frac{\% \text{Lignin after biomass pretreatment}}{\text{\%Lignin original biomass}}\end{matrix}$$

### Process Consolidation and Scale Up

The integration and consolidation of the major unit operations required for converting biomass into biofuels into a single vessel is a significant process improvement strategy that is necessary to improve process economics and efficiencies. However, downstream processes are typically intolerant to high loading of organic solvents. Therefore, the addition of water into the system as a co-solvent with the alkanolamine was studied. Pretreatment experiments were carried out while varying the amount of organic solvent used in the presence of water [100, 75, 50. 25, 5] wt. %. Once an optimal range was identified the process was finally scaled up to test the performance of the process (one-pot pretreatment and saccharification) in an industrial scale pressurized reactor. Biomass pretreatment parameters were adapted from the optimized conditions identified. In a typical experiment, the biomass and pretreatment solvent were loaded into a 1 L 4520 Parr bench top reactor (Parr Instrument Company. Moline, IL) equipped with three-arm, self-centering anchor with PTFE wiper blades.

Initial biomass loading was 40 g in the 1 L vessel and the pretreatment vessels were loaded with 40 wt% biomass and 60 wt% liquid fractions, with the liquid fraction consisting of 75% DI water and 25% 2-aminoethan-1-ol. During pretreatment, the reaction vessels were heated to a reaction temperature of 100° C. for 1 hr under completely mixed conditions. Subsequently, the reactors were cooled and adjusted to pH 5 with 5 M H₂SO₄. Next additional DI water was added to reach 15 wt% solids as measured by the initial solids loading. An enzyme mixture of Cellic® CTec3 and HTec3 at ratio of 9:1 v/v, respectively, was used at a total enzyme loading of 10 mg/g biomass dosing. The reaction vessels were completely mixed and heated to 50° C. for 72 hours. Once complete, the mixture was filtered using a 0.22 um screen and the liquid fraction was reserved for bioconversion studies and also characterized for sugars, acids, phenolics and furans. On the other hand, the solid fraction was recovered for lignin analysis (see below). The recovered lignin extract, i.e., the residual solid after pretreatment and enzymatic hydrolysis (in a one pot setting), was then cleansed and purified to minimize the presence of residual sugars, phenolics or organic solvent. The recovered solid fraction was water washed and returned to a neutral pH, subsequently the lignin was enzymatically treated to ensure complete removal of any polysaccharides. Finally, the recovered solid was cleansed again, centrifuged and lyophilized to recover a sugar-free lignin powder.

### Structural Characterization of Biomass Residue Characterization

PXRD: The raw and pretreated biomass were dried and characterized using powder X-ray diffraction (PXRD). The XRD analyses were performed on a Bruker D2 Phaser (Bruker Scientific Instruments, Billerica, MA) and operated at at 30 kV and 10 mA using Cu K-alpha: λ =1.541 84 A. 1.0 mm Soller slit input, 3 mm knife edge on sample, 2.5 mm Soller slit in front of silicon strip detector opened to 4.75 degrees The patterns were collected in the 2θ range from 5 to 60° with a step size of 0.039° and the exposure time of 300 seconds. A reflection-transmission spinner was used as a sample holder and the spinning rate was set at 8 rpm throughout the experiment. According to previously defined diffractogram, the Bragg angles of peak (110), (1I̅0), (020), and (004) belonging to cellulose I are ~ [14.8°, 16.3°, 22.3°, and 34.5°], respectively. The Bragg angle of the amorphous peak is around 19.5 - 20.5°.¹ The crystallinity index was also calculated according to the method of Segal et.al., where the ratio of the height of the 002 peak (I₀₀₂) and the height of the minimum (I_{AM}) between the 002 and the 101 peaks.^{1,2} The peak deconvolution of the resulting diffractogram was also performed using software PeakFit (SeaSolve Software Inc.). Gaussian/Lorentzian functions were applied in curve fitting analysis and iterations were repeated until the maximum F number was obtained. In all cases, the F number was >10,000, which corresponds to a R² value > 0.99. Estimation of the content amorphous cellulose in the cellulosic samples was established by using the relative peak areas.

Thermal Gravimetric Analysis (TGA): Thermal analysis was determined conducted using a Mettler Toledo TGA/DSC1 unit (Mettler Toledo, Leicester, UK) under N₂ atmosphere (50 mL/min). Samples between 10-20 mg were placed in alumina crucibles (150 µL) and heated from ambient temperature to 600° C. at a heating rate of 10° C./min. The data was analyzed using STARe Evaluation software.

FTIR Analyses: FT-IR spectra were acquired using a Bruker VERTEX 70 system (Billerica, MA) within the range of 4000 to 600 cm⁻¹, resolution of 4 cm⁻¹ and 32 s scan time. The data was analyzed using OPUS (version 8.2) software.

NMR Analysis: The lignin extract recovered after 2-aminoethan-1-ol pretreatment was solubilized in DMSO-d6/pyridine and then analyzed by two-dimensional (2D) ¹³C-¹H heteronuclear single quantum coherence (HSQC) nuclear magnetic resonance (NMR). Briefly, the lignin sample (~50 mg) was solubilized in -600 µL DMSO-d6/pyridine-d5 (2/1 v/v). The samples were sealed and sonicated until homogeneous in a Branson 2510 table-top cleaner Branson Ultrasonic Corporation, Danburt, CT). The temperature of the bath was closely monitored and maintained below 50° C. After complete solubilization, the samples were transferred into NMR tubes. 2D HSQC spectra were recorded on a Bruker Avance I spectrometer operating at 800 MHz that was equipped with a TXI probe at 298 K. ¹H-¹³C correlations were obtained using the Q_HSQC method of Heikkinen et. al.,³ The ¹H and ¹³C spectral widths were set to 13.3 ppm and 160 ppm respectively, with carrier frequencies set to 5 ppm (¹H) and 80 ppm (¹³C). A total of 256 scans were collected for each of 256 blocks, using a recycle delay of 1 sec. Chemical shifts were referenced to the central DMSO peak (δC/δH 39.5/2.5 ppm). Assignment of the HSQC spectra is described elsewhere.⁴⁻⁶ A semiquantitative analysis of the volume integrals of the HSQC correlation peaks was performed using MestReNova (Mestrelab Research S.L.) processing software, version 14.1.2-25024.

### Results and Discussion

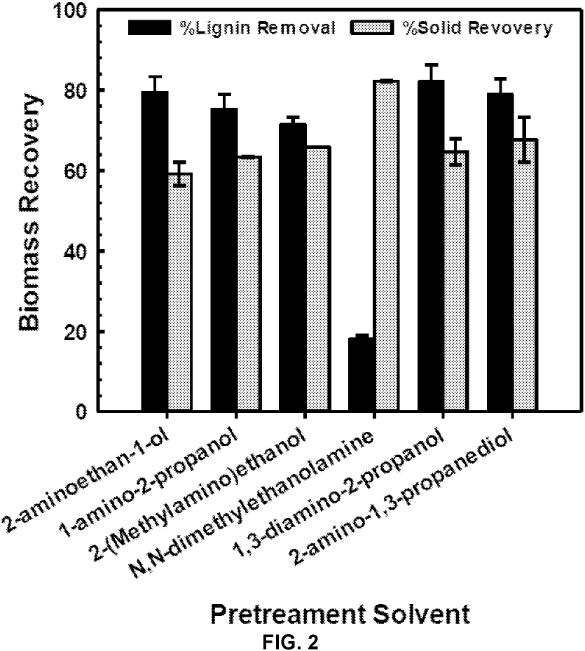

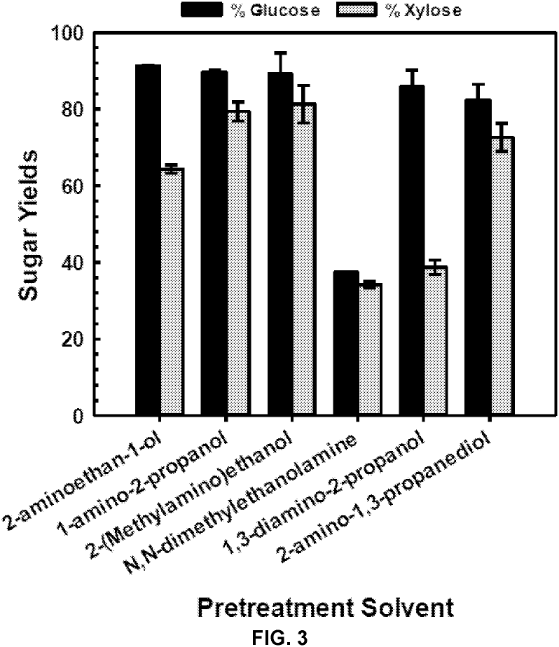

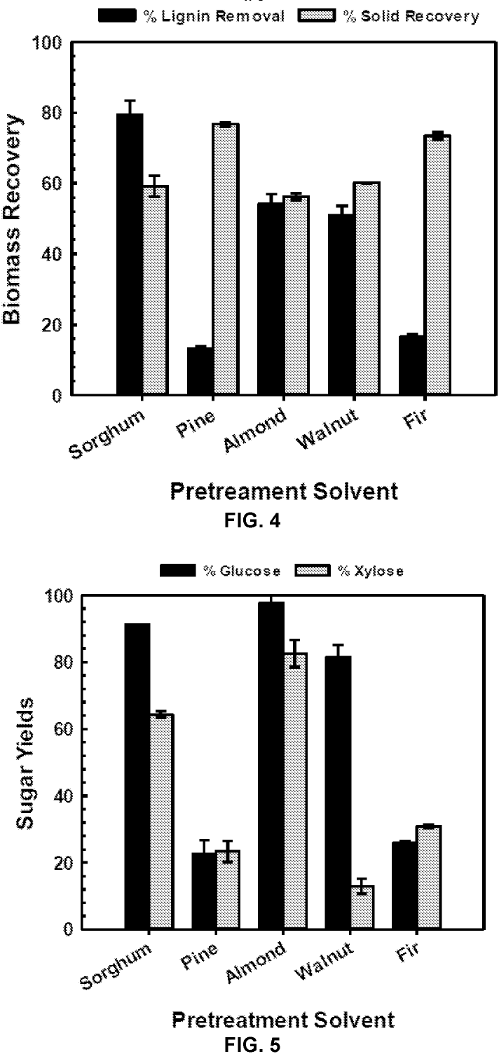

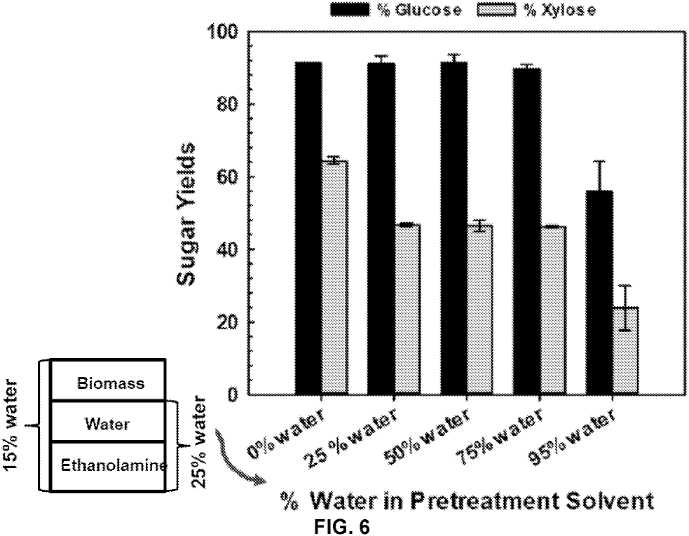

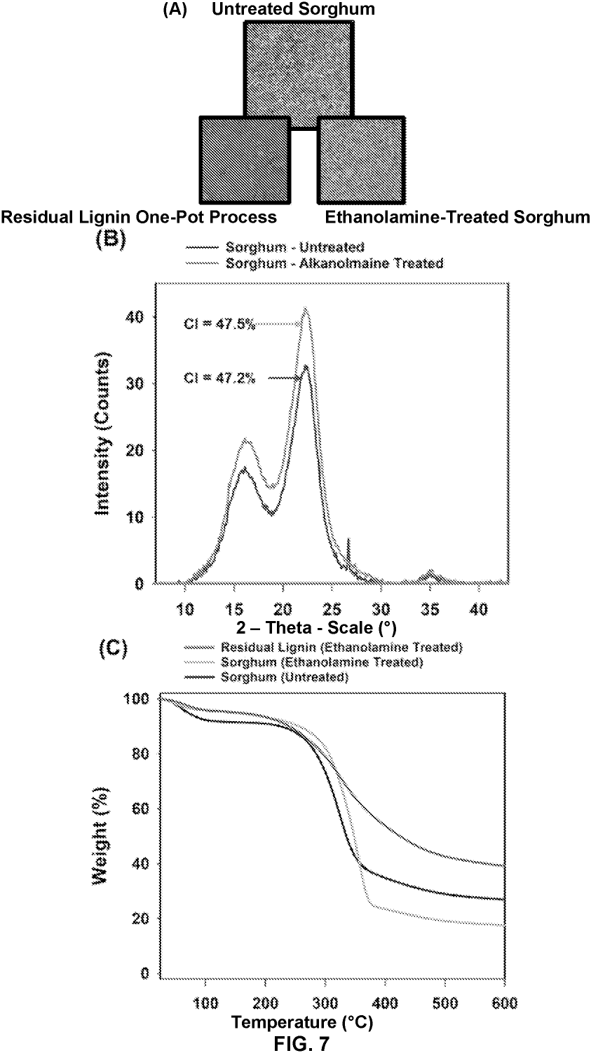

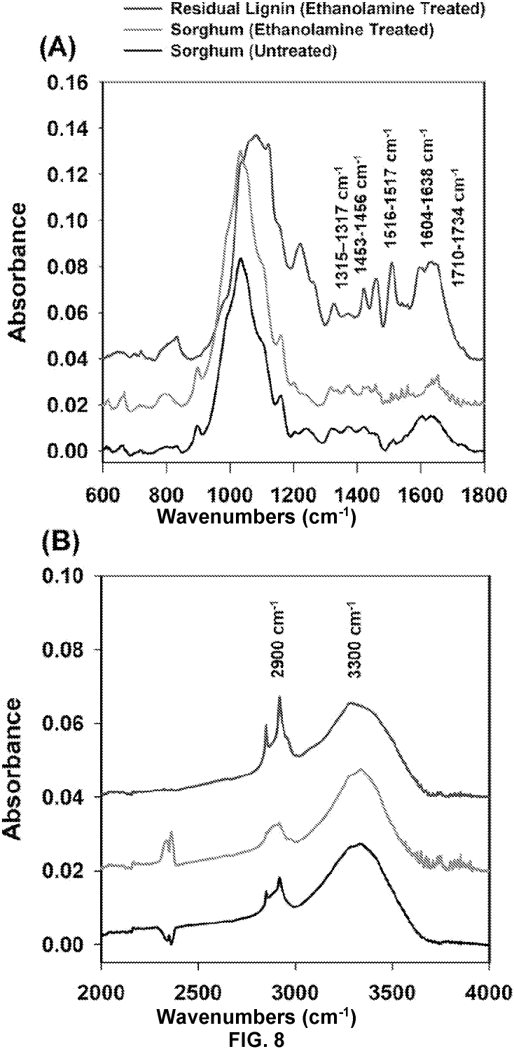

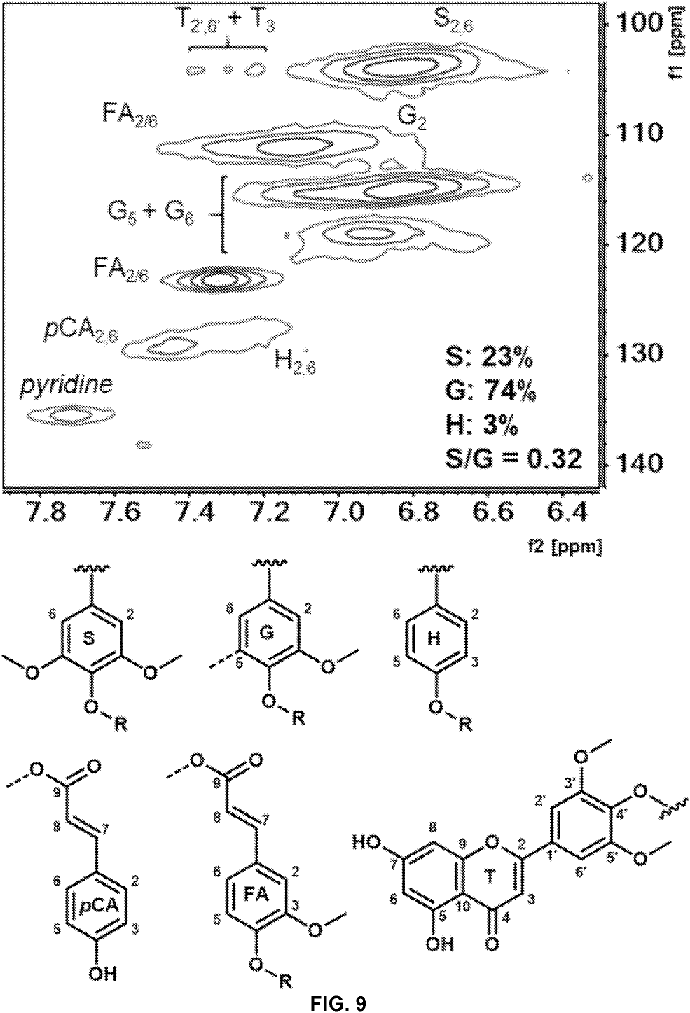

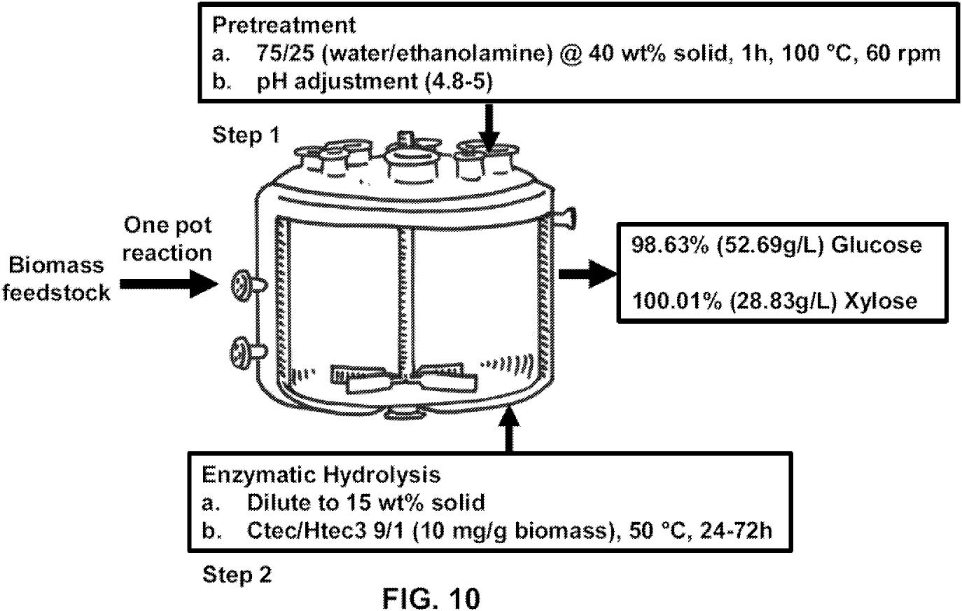

This work demonstrates the feasibility of applying dual functional solvents called alkanolamines towards the conversion of biomass to biofuels. Several key factors were considered to effectively integrate the pretreatment technology into a biorefinery, including solvent screening, effectiveness on a broad range of feedstocks, fractionation of lignocellulose components, enzymatic compatibility. Several molecular solvents with similar functionalities were studied for their pretreatment effectiveness, in terms of lignin/hemicellulose removal and enzymatic hydrolysis yield (FIG. 1B). The results show that both amine and hydroxyl functionality play an important role in controlling lignin removal, however, the amine group was more important. Simple changes in amine functionality (based on number of amines, degree of substitution, and confirmation) proved to significantly influence pretreatment effectiveness (FIGS. 2-3). The lower pretreatment capability of the tertiary amine (N,N-dimethylethanolamine) is due to the reduced polarity, which leads to a weaker interaction between lignin and tertiary amines. In addition, amine dominated solvents (1,3-diamino-2-propanol) had stronger affinity to interact with hydrogen bond donor groups of lignin than the hydroxyl dominated solvent (2-amino-1,3-propanediol). 2-aminoethan-1-ol (or ethanolamine) was evaluated for its effectiveness at pretreating various biomass types, which revealed that sorghum (grassy) along with hardwoods (almond, walnut) are easier to deconstruct than softwood (pine, fir) (FIGS. 4-5). Sorghum was selected for further process optimization due to its high glucan content by screening three factors (pretreatment time, temperature, and solid loading). The results were not significant within the range tested for glucose yields, therefore, a low severity process parameters were identified, and it was found that the pretreatment process can tolerate high water content (up to 75%) (FIG. 6), both of which enable process consolidation and intensification to reduce costs. The pretreated biomass and recovered lignin were studied using PXRD/TGA/NMR analysis and the characterization revealed that the morphology and crystallinity of biomass does not change after pretreatment, and recovered lignin is dominated by guaiacyl groups (FIGS. 7-9). This lignin is less condensed compared to other pretreated lignins, so should be amenable to catalytic upgrading into valuable products. With the final selected conditions, the pretreatment was scaled up, resulting in nearly 100% deconstruction efficiency for enzymatic hydrolysis (FIG. 10). Overall, this demonstrates the effectiveness and robustness of alkanolamines for use in economic biomass pretreatment and presents a new solvent group to be considered for use within commercial biorefineries.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method to produce a sugar compound from a biomass, the method comprising:
(a) providing a first mixture comprising a solubilized biomass and an alkanolamine;
(b) recovering at least part of the alkanolamine from the first mixture in order to separate the at least part of the alkanolamine from the first mixture;
(c) optionally introducing an enzyme and/or a microbe to the first mixture such that the enzyme and/or microbe produce a sugar from the solubilized biomass; and,
(d) optionally the sugar is separated from the first mixture.

2. The method claim 1, wherein the providing step (a) comprises incubating the first mixture at about 100° C. to about 160° C. for at least about 30 minutes.

3. The method claim 1, wherein the recovering step (b) comprises distilling the at least part of the alkanolamine from the first mixture.

4. The method claim 1, further comprising (e) introducing at least part of the alkanolamine separated in the (b) recovering step to the first mixture in step (a).

5. The method claim 1, further comprising (f) introducing more biomass to the first mixture in step (a).

6. The method of claim 1, wherein the alkanolamine comprises at least 3 carbon atoms.

7. The method of claim 6, wherein the alkanolamine has the following structure: 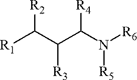
wherein R₁ to R₆ are each independently ——H, ——NH₂, alkyl, alkenyl, alkynyl, aryl, alkyl amine, alkenyl amine, alkynyl amine, or aryl amine, and R₁ to R₄ are each independently —OH, alkanol, alkenol, alkynol, or aryl alkanol, wherein at least one of R₁ to R₄ is —OH, alkanol, alkenol, alkynol, or aryl alkanol.

8. The method of claim 1, wherein the alkanolamine is 2-aminoethan-1-ol, 1-amino-2-propanol, 2-(Methylamino)ethanol, N,N-dimethylethanolamine. 1,3-diamino-2-propanol, or 2-amino-1,3-propanediol.

9. A first mixture comprising a biomass and an alkanolamine comprising at least 3 carbon atoms.

10. The first mixture of claim 9, wherein the alkanolamine has the following structure: 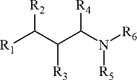
wherein R₁ to R₆ are each independently —H. —NH₂, alkyl, alkenyl, alkynyl, aryl, alkyl amine, alkenyl amine, alkynyl amine, or aryl amine, and R₁ to R₄ are each independently —OH, alkanol, alkenol, alkynol, or aryl alkanol, wherein at least one of R₁ to R₄ is —OH, alkanol, alkenol, alkynol, or aryl alkanol.

11. The first mixture of claim 9, wherein the alkanolamine is a 1-amino-2-propanol, 2-(methylamino)ethanol. N,N-dimethylethanolamine, 1,3-diamino-2-propanol, or 2-amino-1,3-propanediol.

12. A first mixture comprising a biomass and an alkanolamine having a ratio of more 1:19 by volume or weight.

13. The first mixture of claim 11, wherein the ratio is equal or more than 1:10 by volume or weight.

14. The first mixture of claim 12, wherein the alkanolamine has the following structure: 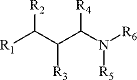
wherein R₁ to R₆ are each independently ——H, ——NH₂, alkyl, alkenyl, alkynyl, aryl, alkyl amine, alkenyl amine, alkynyl amine, or aryl amine, and R₁ to R₄ are each independently —OH, alkanol, alkenol, alkynol, or aryl alkanol, wherein at least one of R₁ to R₄ is —OH, alkanol, alkenol, alkynol, or aryl alkanol.

15. The first mixture of claim 12, wherein the alkanolamine is a 2-aminoethan-1-ol, 1-amino-2-propanol, 2-(methylamino)ethanol, N.N-dimethylethanolamine, 1.3-diamino-2-propanol, or 2-amino-1,3-propanediol.
